(19) United States
(12) Patent Application Publication
(10) Pub. No.: US 2025/0134888 A1
(43) Pub. Date: May 1, 2025

(54) INHIBITORS OF MALIC ENZYME 1

(71) Applicants: Cornell University, Ithaca, NY (US); The Regents of the University of Michigan, Ann Arbor, MI (US)

(72) Inventors: Lewis Cantley, Ithaca, NY (US); Costas Lyssiotis, Ann Arbor, MI (US); Lin Lin, Ann Arbor, MI (US); Mengrou Shan, Ann Arbor, MI (US); Osamu Kaneko, Tokyo (JP); Akio Kamikawa, Tokyo (JP)

(21) Appl. No.: 18/836,654

(22) PCT Filed: Feb. 7, 2023
(86) PCT No.: PCT/US2023/062139

§ 371 Date: Aug. 7, 2024

Related U.S. Application Data
(60) Provisional application No. 63/307,488, filed on Feb. 7, 2022.

(51) Int. Cl. A61K 31/5025; A61K 31/444; A61K 31/4545; A61K 31/4709; A61K 31/519; A61P 35/00; C07D 213/69; C07D 401/06; C07D 471/04; C07D 487/04; C07D 519/00

(52) U.S. Cl. CPC A61K 31/5025; A61K 31/444; A61K 31/4545; A61K 31/4709; A61K 31/519; A61P 35/00; C07D 213/69; C07D 401/06; C07D 471/04; C07D 487/04; C07D 519/00

(57) ABSTRACT

The present disclosure relates to compounds that inhibit malic enzyme 1. The disclosure further relates to methods of treating cancers mediated by malic enzyme 1.

### RELATED APPLICATION

This application claims the benefit of and priority to U.S. Provisional Patent Application Ser. No. 63/307,488, filed Feb. 7, 2022, which is hereby incorporated by reference in its entirety.

### BACKGROUND

Pancreatic ductal adenocarcinoma (PDA) is projected to become the second most common cause of cancer death in the United States by 2030, in spite of a relatively low incidence rate (Rahib et al. 2014). The lethality of PDA is due largely to the lack of effective treatment options at all stages Targeted therapy and immunotherapy, while showing promise for other cancers, has thus far proven ineffective for PDA (Halbrook et al. 2017; Brahmer et al. 2012). Clearly new therapeutic approaches and drug targets are needed.

A subset of PDA tumors lose the tumor suppressor SMAD4 during progression. The metabolic enzyme malic enzyme 2 (ME2) is located in close genomic proximity to SMAD4. Consequently, ME2 is lost at high frequency in SMAD4 deleted tumors, creating a context for targeting other ME isoforms, known as collateral lethality.

### SUMMARY OF THE INVENTION

The present disclosure provides compounds represented by formula (IA):

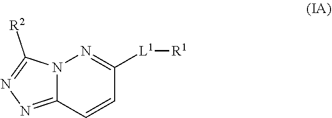

wherein
L¹ is a single bond connecting to R¹, or is —C(O)NH— or —NHC(O)—;
R¹ is aryl, heteroaryl, or heterocyclyl; and
R² is aryl, heteroaryl, or heterocyclyl,
or a pharmaceutically acceptable salt thereof.

The present disclosure also provides compounds represented by formula (IB):

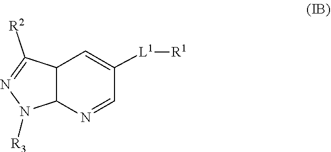

wherein
L¹ is a single bond connecting to R¹, or is —C(O)NH— or —NHC(O)—;
R¹ is aryl, heteroaryl, or heterocyclyl;
R² is alkyl, aryl, heteroaryl, or heterocyclyl; and
R³ is aryl or heteroaryl,
or a pharmaceutically acceptable salt thereof.

The present disclosure also provides compounds represented by formula (IC):

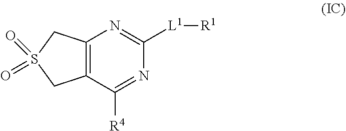

wherein
L¹ is a single bond connecting to R¹, or is —C(O)NH— or —NHC(O)—;
R¹ is aryl, heteroaryl, or heterocyclyl; and
R⁴ is a heterocyclyl,
or a pharmaceutically acceptable salt thereof.

The present disclosure further provides a compound represented by formula (II):

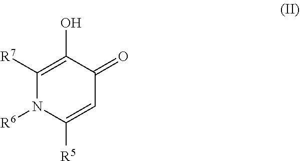

wherein
R⁵ is alkyl;
R⁶ and R⁷ are, independently, H, alkyl, -L²-L³-R⁸, or -L²-R⁸;
R⁸ is aryl, heteroaryl, or heterocyclyl;
L² is alkylene or heterocyclyl; and
L³ is heterocyclyl;
provided that one and only one of R⁶ and R⁷ is -L²-L³-R⁸ or -L²-R⁸.
or a pharmaceutically acceptable salt thereof.

In certain embodiments, the present disclosure provides pharmaceutical compositions comprising a compound provided herein and a pharmaceutically acceptable excipient.

In certain embodiments, the present disclosure provides methods of treating a cancer in a subject in need thereof, the method comprising administering a therapeutically effective amount of a compound provided herein to the subject.

In certain embodiments, the present disclosure provides methods of treating a cancer in a subject in need thereof, the method comprising administering to the subject a therapeutically effective amount of an agent that downregulates the copy number, amount, and/or activity of ME1.

### BRIEF DESCRIPTION OF THE DRAWINGS

FIG. 1: Genomic proximity of ME2 and SMAD4 and subcellular expression of ME isoforms.

FIG. 2: Synthetic lethal context for ME1 inhibition imposed by SMAD4/ME2 genetic loss. Figure adapted from Muller et al. 2015.

FIG. 3A: Genetic inhibition of ME1 is growth inhibitory in some PDA cell lines and specimens. Representative colony formation assay (CFA) data for two ME1 knockdown responder lines (left) and ME1 knockdown non-responder lines (right). Cell lines expressing two independent ME1 doxycycline (Dox)-inducible shRNAs (ME1sh1 and ME1sh3) are presented in the columns.

FIG. 3B: Genetic inhibition of ME1 is growth inhibitory in some PDA cell lines and specimens. Data are plotted as the relative number of colonies upon Dox-induced ME1 knockdown, relative to mock-treated isogenic controls. Primary patient-derived PDA cultures established at the University of Michigan are labeled as UM# or UMich-#.

FIG. 3C. ME1 knockout with sgME1/Crispr-Cas9 reproduces the colony forming assay response with shME1. ME1 responder PDAC cell lines Tu8902 and MIA PaCa2 exhibit a significant reduction in colony formation following sgME1 or triple knockout targeting the three ME isoforms (sgME1/sgME2/sgME3), compared to non-targeting (NT) control. 8988T is an intermediate responder PDAC cell line, and YAPC is a non-responder cell line.

FIG. 4A: Final tumor volume of UM2 primary patient-derived xenograft tumors containing a Dox-inducible (iDox) NT hairpin (Non-target control), treated−/+Dox. Dox administration was initiated at day 7.

FIG. 4B: Inhibition of ME1 blocks tumor growth in pancreatic patient-derived xenografts (PDX). Final tumor volume of UM2 primary patient-derived xenograft tumors containing a Dox-inducible hairpin targeting ME1, treated−/+Dox. Dox administration was initiated at day 7.

FIG. 4C: Inhibition of ME1 regresses established pancreatic patient-derived xenografts (PDX). Dox was administered to animals bearing established UM2 PDX tumors that contain iDox-shME1 at day 23 (schedule A, 100 mm³ tumors) or day 37 (schedule B, 250 mm³ tumors).

FIG. 4D: Immunohistochemical and H&E staining of UM2 PDX tumors that contain iDox-shME1 and treated−/+Dox.

FIG. 5: ME1 inhibition sensitive (responder) PDA cell lines and specimens exhibit no/low ME2 expression, relative to non-responders.

FIG. 6A: Single cell clonal ME2 knockout by Crispr-Cas9 in PANC-1. Clonal lines were selected and validated via Western blots. BxPC3 cells are ME2 null and serve as control.

FIG. 6B: Pooled Crispr-Cas9 ME2 knockout PANC-1 and TU8902 sub-lines, of which 17 different guide-RNA oligos are transduced and screened by Western blot. Parental (wild type) “WT” cells serve as controls for the densitometry analysis and ME2 protein reduction in the knockout sub-lines are shown as the ratios normalized to the loading control vinculin (VCL).

FIG. 7A: ME2 KO in ME1-non responder PDA cell line induces ME1 dependence. CRISPR/Cas9 knockout of ME1, ME2, or double knockout of ME1 and ME2 in 2 representative non-responder cell lines, HupT3 and Panc03.27. CFA data are plotted as the relative number of colonies to non-target CRISPRs (sgNT), which are relative to mock-treated isogenic controls.

FIG. 7B: Western blotting analysis of the pooled CRISPR/Cas9 knockout of ME1 and ME2, or double knockout of ME1 and ME2 in 2 representative non-responder cell lines, HupT3 and Panc03.27.

FIG. 8A: Characterization of ER stress and mitochondrial health upon inhibition of ME1. Western blotting of biomarkers of mitochondrial morphology and ER homeostasis.

FIG. 8B: Top-Confocal microscopic imaging of TU8902 following ME1 knockdown (KD) using MitoSpy (green) and Hoechst (blue); Bottom—ME1 knockdown using TMRE (red) and Hoechst (blue). (60× water lens).

FIG. 8C: Quantitation of flow cytometry of TU8902 stained with TMRE and cell viability dye Sytox Blue (left, middle). Seahorse measurement of oxygen consumption rate (OCR) following ME1 knockdown (right).

FIG. 8D: Quantitation of flow cytometry of PANC-1 with TMRE and Sytox Blue (left, middle). Seahorse measurement of OCR following ME1 knockdown (right). Positive controls: FCCP uncouples and therefore depolarizes mitochondria; Oligomycin (Oligo) is a complex V inhibitor that promotes membrane potential.

FIG. 9: Allosteric and active sites of ME1.

FIG. 10: Model of x-ray co-crystal structure with inhibitor.

### DETAILED DESCRIPTION OF THE INVENTION

Pancreatic cancer is the deadliest major cancer with a 5-year survival rate below 10%. This is attributed largely to the lack of therapeutic options. Pancreatic cancers with genomic loss of the tumor suppressor SMAD4 co-delete the house-keeping metabolic enzyme, malic enzyme 2 (ME2), due to the proximity of these genes on chromosome 18. This creates a synthetic lethal context for inhibition of the ME1 isoform. Namely, ME1 inhibition in ME2 null pancreatic cancer cells and xenograft tumors leads to profound growth inhibition, and in some cases, tumor regression with ME1 knockdown as a single agent.

Further, normal human cells tolerate ME1 inhibition, and germline Me1 knockout mice are born healthy, mature to adulthood without observable phenotypes, and produce viable offspring. Similarly, Me1 knockout in adult animals, an experimental model that more closely mimics ‘drug’ treatment, is well tolerated.

The mechanism is further elucidated in Yoshida et al. 2022, which is hereby incorporated by reference.

It has been demonstrated that ME2 deleted tumors are sensitized to ME1 inhibition using gene silencing with shRNA and knockout with Crispr-Cas9. ME2 low expressing tumor models are similarly sensitized, but ME2 proficient tumors are resistant to ME1. This paradigm is evident in cell line and tumor models. Given this genetic liability, small molecule inhibitors of MEs were discovered. And, furthermore, based on these collective data, lack of ME2 expression can be used as a patient-specific biomarker to deploy ME1 inhibitors.

### Compounds

The present disclosure provides compounds represented by formula (IA):

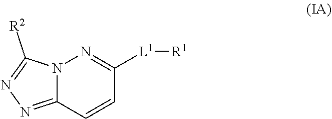

wherein
L¹ is a single bond connecting to R¹, or is —C(O)NH— or —NHC(O)—;
R¹ is aryl, heteroaryl, or heterocyclyl; and
R² is aryl, heteroaryl, or heterocyclyl,
or a pharmaceutically acceptable salt thereof.

In certain embodiments, L¹ is a single bond to R¹.

In certain embodiments, R¹ is substituted heterocyclyl.

In certain embodiments, R¹ is an alkyl substituted piperidinyl.

In certain embodiments, R¹ is

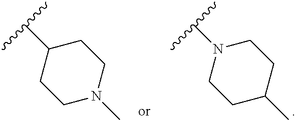

In certain embodiments, R² is substituted aryl or heteroaryl.

In certain embodiments, R² is a phenyl substituted with at least one R^{2a} selected from halo, alkylamino, alkylaminoalkyl, and —NHC(O)R^{2b}, wherein R^{2b} is a substituted heterocyclyl.

In certain embodiments, R² is a phenyl substituted with one R^{2a} selected from alkylaminoalkyl and —NHC(O)R^{2b}, wherein R^{2b} is a substituted heterocyclyl.

In certain embodiments, R^{2b} is an alkyl substituted piperidinyl.

In certain embodiments, the substituted phenyl is substituted at the 3 or 4 position of the phenyl group.

In certain embodiments, the compound having the structure:

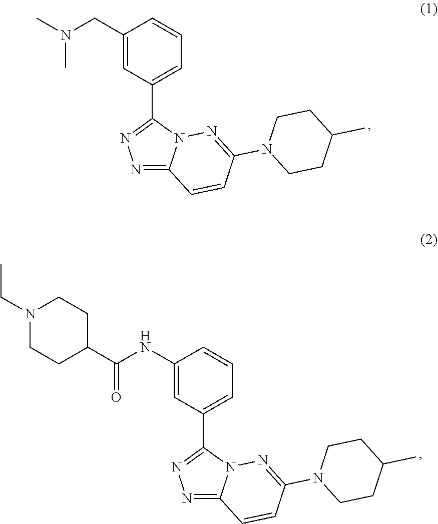

or a pharmaceutically acceptable salt thereof.

The present disclosure provides compounds represented by formula (IB):

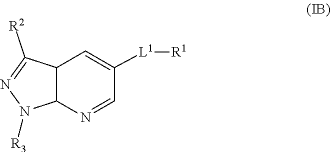

wherein
L¹ is a single bond connecting to R¹, or is —C(O)NH— or —NHC(O)—;
R¹ is aryl, heteroaryl, or heterocyclyl;
R² is alkyl, aryl, heteroaryl, or heterocyclyl; and
R³ is aryl or heteroaryl,
or a pharmaceutically acceptable salt thereof.

In certain embodiments, L¹ is —C(O)NH—.

In certain embodiments, R¹ is substituted heterocyclyl.

In certain embodiments, R¹ is an alkyl substituted piperidinyl.

In certain embodiments, R¹ is

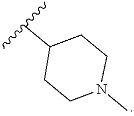 [SMILES: *C1CCN(C)CC1]

In certain embodiments, R² is alkyl.

In certain embodiments, R² is C₁-C₆ alkyl.

In certain embodiments, R³ is substituted aryl or heteroaryl.

In certain embodiments, R³ is a phenyl substituted with at least one R^{3a} selected from halo, alkylamino, alkylaminoalkyl, and —NHC(O)R^{3b}, wherein R^{3b} is a substituted heterocyclyl.

In certain embodiments, R³ is a phenyl substituted with one R^{3a} selected from a halo.

In certain embodiments, the substituted phenyl is substituted at the 3 or 4 position of the phenyl group.

In certain embodiments, the compound having the structure:

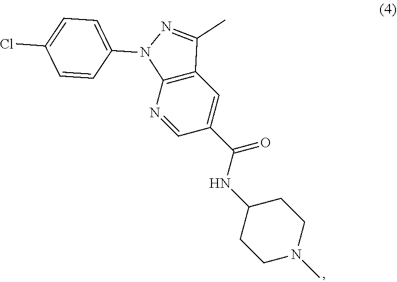 [SMILES: Cc1nn(-c2ccc(Cl)cc2)c2ncc(C(=O)NC3CCN(C)CC3)cc12]

or a pharmaceutically acceptable salt thereof.

The present disclosure provides compounds represented by formula (IC):

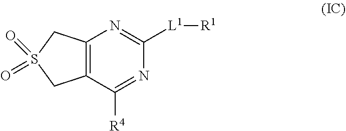

wherein
L¹ is a single bond connecting to R¹, or is —C(O)NH— or —NHC(O)—;
R¹ is aryl, heteroaryl, or heterocyclyl; and
R⁴ is a heterocyclyl,
or a pharmaceutically acceptable salt thereof.

In certain embodiments, L¹ is absent.

In certain embodiments, R¹ is substituted heterocyclyl.

In certain embodiments, R¹ is an alkyl substituted piperidinyl.

In certain embodiments, R¹ is

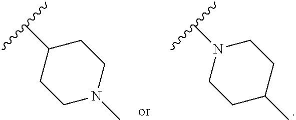

In certain embodiments, R⁴ is a fused bicyclic heteroaryl.

In certain embodiments, R⁴ is a [6.5] fused bicyclic heteroaryl comprising at least one nitrogen.

In certain embodiments, R⁴ is a [6.5] fused bicyclic heteroaryl comprising three nitrogens.

In certain embodiments, R⁴ is

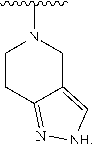 [SMILES: *N1CCc2n[nH]cc2C1]

In certain embodiments, the having the structure:

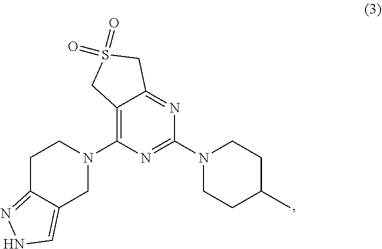 [SMILES: CC1CCN(c2nc3c(c(N4CCc5n[nH]cc5C4)n2)CS(=O)(=O)C3)CC1]

or a pharmaceutically acceptable salt thereof.

The present disclosure provides compounds represented by formula (II):

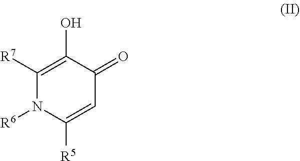

wherein
R⁵ is alkyl;
R⁶ and R⁷ are, independently, H, alkyl, -L²-L³-R⁸, or -L²-R⁸;
R⁸ is aryl, heteroaryl, or heterocyclyl;
L² is a single bond to L³ or R⁸, or is alkylene or heterocyclyl; and
L³ is a single bond to R⁸, or is heterocyclyl;
provided that one of R⁶ and R⁷ is -L²-L³-R⁸ or -L²-R⁸,
or a pharmaceutically acceptable salt thereof.

In certain embodiments, the compound is not

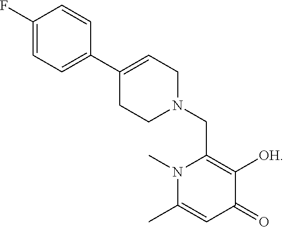 [SMILES: Cc1cc(=O)c(O)c(CN2CC=C(c3ccc(F)cc3)CC2)n1C]

In certain embodiments, the compound having the structure:

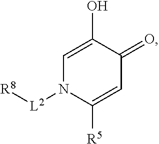

or a pharmaceutically acceptable salt thereof.

In certain embodiments, R⁵ is C₁-C₆ alkyl.

In certain embodiments, R⁵ is —CH₃.

In certain embodiments, L² is C₂-C₆ alkylene.

In certain embodiments, R⁸ is a fused bicyclic heteroaryl.

In certain embodiments, R⁸ is an [6.6] fused bicyclic heteroaryl comprising at least one nitrogen.

In certain embodiments, R⁸ is

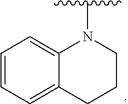 [SMILES: *N1CCCc2ccccc21]

In certain embodiments, R⁸ is substituted aryl or heteroaryl.

In certain embodiments, R⁸ is a phenyl substituted with at least one R^{8a} selected from halo, alkylamino, alkylaminoalkyl, and —NHC(O)R^{8b}, wherein R^{8b} is a substituted heterocyclyl.

In certain embodiments, R⁸ is a phenyl substituted with one R^{8a} selected from a halo.

In certain embodiments, the substituted phenyl is substituted at the 3 or 4 position of the phenyl group.

In certain embodiments, the compound having the structure:

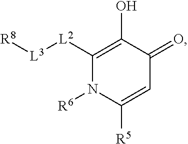

or a pharmaceutically acceptable salt thereof.

In certain embodiments, R⁵ is C₁-C₆ alkyl. In other embodiments, R⁵ is —CH₃.

In certain embodiments, R⁶ is C₁-C₆ alkyl. In other embodiments, R⁶ is —CH₃.

In certain embodiments, L² is C₁-C₆ alkylene.

In certain embodiments, L³ is -piperidinyl- or -tetrahydropyridinyl-.

In certain embodiments, L³ is

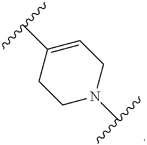 [SMILES: *C1=CCN(*)CC1]

In certain embodiments, R⁸ is substituted aryl or heteroaryl.

In certain embodiments, R⁸ is a phenyl substituted with at least one R^{8a} selected from halo, alkylamino, alkylaminoalkyl, and —NHC(O)R^{8b}, wherein R^{8b} is a substituted heterocyclyl.

In certain embodiments, R⁸ is a phenyl substituted with one R^{8a} selected from a halo.

In certain embodiments, the substituted phenyl is substituted at the 3 or 4 position of the phenyl group.

In certain embodiments, the having the structure

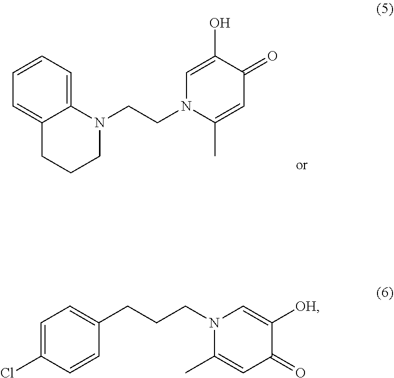 [SMILES: Cc1cc(=O)c(O)cn1CCN1CCCc2ccccc21 ; Cc1cc(=O)c(O)cn1CCCc1ccc(Cl)cc1]

or a pharmaceutically acceptable salt thereof.

In certain embodiments, the having the structure

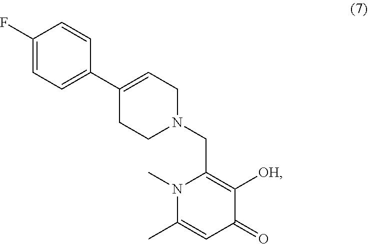 [SMILES: Cc1cc(=O)c(O)c(CN2CC=C(c3ccc(F)cc3)CC2)n1C]

or a pharmaceutically acceptable salt thereof.

The compounds of the present invention may be prepared by techniques known to those skilled in the art. For example, such techniques are described in Vogel's Textbook of Practical Organic Chemistry, A. I. Vogel, A. R. Tatchell, B. S. Furnis, A. J. Hannaford, P. W. G. Smith, (Prentice Hall) 5th Edition (1996) and/or March's Advanced Organic Chemistry: Reactions, Mechanisms, and Structure, Michael B. Smith, Jerry March, (Wiley-Interscience) 5th Edition (2007), and references therein. . . . However, these may not be the only means by which to synthesize or obtain the desired compounds.

The various groups attached to the heteroaryl or heterocyclyl cores of the compounds disclosed herein may be added to the rings by techniques known to those skilled in the art, for example those set forth in Advanced Organic Chemistry Part B: Reaction and Synthesis, Francis Carey and Richard Sundberg, (Springer) 5th ed. Edition. (2007).

### Pharmaceutical Compositions

In certain embodiments, a pharmaceutical composition comprising a compound of the present disclosure, e.g. a compound of formula (IA), (IB), (IC), or (II), and one or more pharmaceutically acceptable excipients.

The compositions and methods of the present disclosure may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the disclosure and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the disclosure. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a self-emulsifying drug delivery system or a self-2microemulsifying drug delivery system. The pharmaceutical composition (preparation) also can be a liposome or other polymer matrix, which can have incorporated therein, for example, a compound of the disclosure. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (or preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the disclosure, with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present disclosure to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this disclosure, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with a compound of the disclosure. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the disclosure will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present disclosure, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the disclosure may be used alone or conjointly administered with another type of therapeutic agent.

The present disclosure includes the use of pharmaceutically acceptable salts of compounds of the disclosure in the compositions and methods of the present disclosure. In certain embodiments, contemplated salts of the disclosure include, but are not limited to, alkyl, dialkyl, trialkyl or tetra-alkyl ammonium salts. In certain embodiments, contemplated salts of the disclosure include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the disclosure include, but are not limited to, Na, Ca, K, Mg, Zn or other metal salts. In certain embodiments, contemplated salts of the disclosure include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Methods of Treatment

In certain embodiments, the present disclosure provides a method of treating a disease or condition, such as a cancer, in a subject in need thereof, comprising administering a therapeutically effective amount of a compound provided herein, e.g. a compound of formula (IA), (IB), (IC), and (II).

In certain embodiments, the present disclosure provides a method of treating a cancer in a subject in need thereof, the method comprising administering to the subject a therapeutically effective amount of an agent that downregulates the copy number, amount, and/or activity of malic enzyme 1 (ME1).

In certain embodiments, the agent is a CRISPR guide RNA (gRNA), RNA interfering agent, antisense oligonucleotide, peptide or peptidomimetic inhibitor, aptamer, antibody, or intrabody.

An “RNA interfering agent” as used herein, is defined as any agent which interferes with or inhibits expression of ME1 by RNA interference (RNAi). Such RNA interfering agents include, but are not limited to, nucleic acid molecules including RNA molecules which are homologous to ME1 gene, or a fragment thereof, short interfering RNA (siRNA), and microRNA (miRNA) which interfere with or inhibit expression of ME1 by RNA interference (RNAi).

“RNA interference (RNAi)” is an evolutionally conserved process whereby the expression or introduction of RNA of a sequence that is identical or highly similar to a target biomarker nucleic acid results in the sequence specific degradation or specific post-transcriptional gene silencing (PTGS) of messenger RNA (mRNA) transcribed from that targeted gene (see Coburn and Cullen (2002) J. Virol. 76:9225), thereby inhibiting expression of the target biomarker nucleic acid. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex that recognizes and cleaves target mRNAs. RNAi can also be initiated by introducing nucleic acid molecules, e.g., synthetic siRNAs or RNA interfering agents, to inhibit or silence the expression of target biomarker nucleic acids. As used herein, “inhibition of ME1 expression” includes any decrease in expression, protein activity, or level of a ME1 nucleic acid or protein encoded by a ME1 nucleic acid. The decrease may be of at least 30%, 40%, 50%, 60%, 70%, 80%, 90%, 95% or 99% or more as compared to the expression of a ME1 nucleic acid or the activity or level of the protein encoded by a ME1 nucleic acid which has not been targeted by an RNA interfering agent.

In addition to RNAi, genome editing can be used to modulate the copy number or genetic sequence of ME1, such as constitutive or induced knockout or mutation of ME1. For example, the CRISPR-Cas system can be used for precise editing of genomic nucleic acids (e.g., for creating non-functional or null mutations). In such embodiments, the CRISPR guide RNA and/or the Cas enzyme may be expressed. For example, a vector containing only the guide RNA can be administered to an animal or cells transgenic for the Cas9 enzyme. Similar strategies may be used (e.g., designer zinc finger, transcription activator-like effectors (TALEs) or homing meganucleases). Such systems are well-known in the art (see, for example, U.S. Pat. No. 8,697,359; Sander and Joung (2014) Nat. Biotech. 32:347-355; Hale et al. (2009) Cell 139:945-956; Karginov and Hannon (2010) Mol. Cell 37:7; U.S. Pat. Publ. 2014/0087426 and 2012/0178169; Boch et al. (2011) Nat. Biotech. 29:135-136; Boch et al. (2009) Science 326:1509-1512; Moscou and Bogdanove (2009) Science 326:1501; Weber et al. (2011) PLoS One 6:e19722; Li et al. (2011) Nucl. Acids Res. 39:6315-6325; Zhang et al. (2011) Nat. Biotech. 29:149-153; Miller et al. (2011) Nat. Biotech. 29:143-148; Lin et al. (2014) Nucl. Acids Res. 42:e47). Such genetic strategies can use constitutive expression systems or inducible expression systems according to well-known methods in the art.

In certain embodiments, the RNA interfering agent is a small interfering RNA (siRNA), CRISPR RNA (crRNA), a small hairpin RNA (shRNA), a microRNA (miRNA), or a piwi-interacting RNA (piRNA). In certain embodiments, the RNA interfering agent is a CRISPR guide RNA (gRNA).

“Short interfering RNA” (siRNA), also referred to herein as “small interfering RNA” is defined as an agent which functions to inhibit expression of a target biomarker nucleic acid, e.g., a ME1 nucleic acid, by RNAi. A siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, or 22 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

In another embodiment, a siRNA is a small hairpin (also called stem loop) RNA (shRNA). In one embodiment, these shRNAs are composed of a short (e.g., 19-25 nucleotide) antisense strand, followed by a 5-9 nucleotide loop, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501 incorporated by reference herein).

Multiple siRNA, shRNA, CRISPR constructs for reducing ME1 expression are available commercially, such as siRNA product #SR^{302851,} and shRNA products #TL³¹¹⁵²⁵ and TL³¹¹⁵²⁵V from Origene Technologies (Rockville, MD), and CRISPR gRNA product #sc-401587, and RNAi products Cat #sc-95470 and #sc-95470-SH from Santa Cruz.

In some embodiments, the agent is an antisense oligonucleotide. For example, an oligonucleotide complementary to the area around ME1 polypeptide translation initiation site can be synthesized. One or more antisense oligonucleotides can be added to cell media, typically at 200 μg/ml, or administered to a patient to prevent the synthesis of ME1 polypeptide. The antisense oligonucleotide is taken up by cells and hybridizes to ME1 mRNA to prevent translation. Alternatively, an oligonucleotide which binds double-stranded DNA to form a triplex construct to prevent DNA unwinding and transcription, binds to ME1 mRNA to block translation, or binds to a splicing junction of the ME1 pre-mRNA to alter splicing can be used. As a result of any of these mechanisms, expression of the full-length of ME1 is reduced or blocked.

In another embodiment, the agent inhibits one or more activities of ME1. In one embodiment, the agent inhibits the interaction of ME1 with its natural binding partner(s). Examples of such inhibitory agents include antisense nucleic acid molecules, anti-ME1 antibodies, and ME1 inhibitors. In certain embodiments, the agent comprises an antibody and/or intrabody, or an antigen-binding fragment thereof, which specifically binds to ME1.

In certain embodiments, the cancer is mediated by malic enzyme 1 (ME1).

In certain embodiments, the cancer is a malic enzyme 2 (ME2)-deficient cancer. The “ME2-deficient cancer” refers to any cancer that comprises a cancer cell that has a lower level of ME2 expression compared to a normal cell of the same type. In some specific embodiments, the “ME2-deficient cancer” lacks ME2 expression.

In certain embodiments, the cancer is a gastrointestinal cancer.

In certain embodiments, the cancer is esophageal cancer, stomach cancer, colorectal cancer, pancreatic cancer, or liver cancer.

In certain embodiments, the cancer is pancreatic ductal adenocarcinoma (PDA).

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well-known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known. The ability of such agents to inhibit ME1 may render them suitable as “therapeutic agents” in the methods and compositions of this disclosure.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. As used herein, and as well understood in the art, “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

The terms “cancer” or “tumor” or “hyperproliferative” refer to the presence of cells possessing characteristics typical of cancer-causing cells, such as uncontrolled proliferation, immortality, metastatic potential, rapid growth and proliferation rate, and certain characteristic morphological features. In some embodiments, such cells exhibit such characteristics in part or in full due to the expression and activity of ME1.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present disclosure can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amide”, as used herein, refers to a group

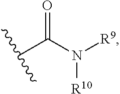

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

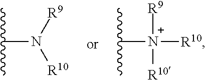

wherein R⁹, R¹⁰, and R^{10′} each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

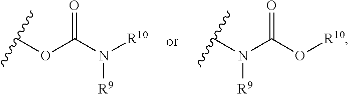

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo[2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo[4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamide” is art-recognized and refers to the group represented by the general formulae

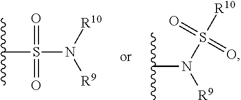

wherein R⁹ and R¹⁰ independently represents hydrogen or hydrocarbyl.

The term “sulfone” is art-recognized and refers to the group represented by the general formulae

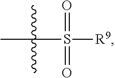

wherein R⁹ represents hydrogen or hydrocarbyl.

The term “sulfoxide” is art-recognized and refers to the group —S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹
wherein R⁹ represents a hydrocarbyl.

The term “thioether”, as used herein, is equivalent to an ether, wherein the oxygen is replaced with a sulfur.

The term “urea” is art-recognized and may be represented by the general formula

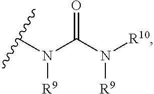

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Some of the compounds may also exist in tautomeric forms. Such forms, although not explicitly indicated in the formulae described herein, are intended to be included within the scope of the present disclosure.

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. No. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “LogS” or “logS” as used herein is used in the art to quantify the aqueous solubility of a compound. The aqueous solubility of a compound significantly affects its absorption and distribution characteristics. A low solubility often goes along with a poor absorption. LogS value is a unit stripped logarithm (base 10) of the solubility measured in mol/liter.

### Discussion

### Malic Enzyme 1

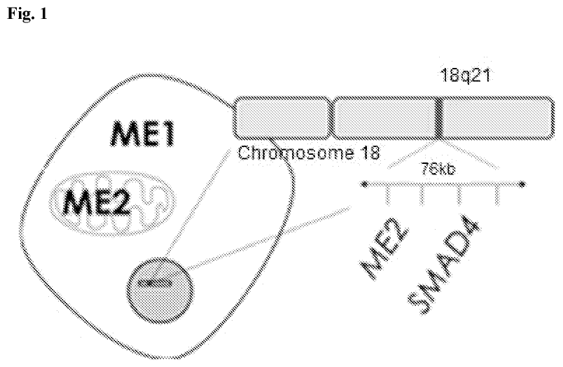

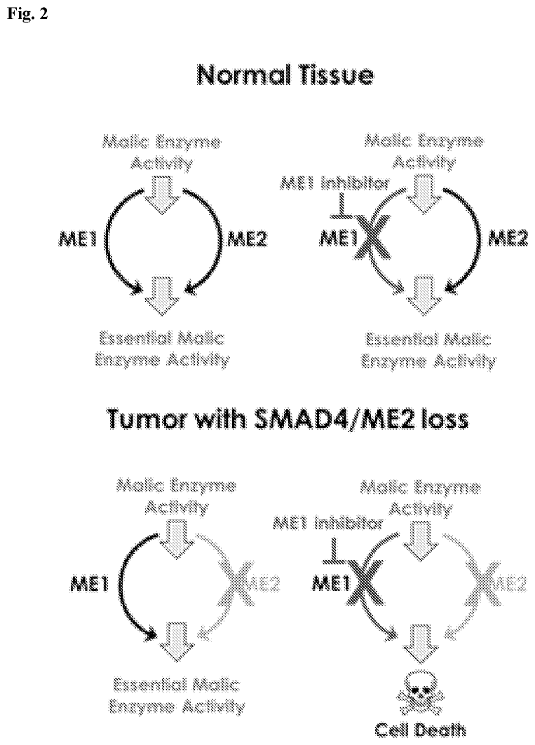

ME1 is cytosolic and has two mitochondrial isoforms, ME2 and ME3. By western blotting for ME isoform expression, clear inverse correlation was found between ME2 expression and ME1 knockdown sensitivity. The ME2 gene is on chromosome 18q21 in close proximity to the tumor suppressor gene SMAD4 (FIG. 1). SMAD4/ME2 loss is observed in ˜20% of PDAs and >6% of other gastrointestinal malignancies, including colon, esophagus, biliary, and stomach. These results suggest that there is functional redundancy between cytosolic ME1 and mitochondrial ME2, where genomic loss of ME2 creates a synthetic lethal context to target ME1 (FIG. 2). Moreover, these results indicate that low/absence of ME2 expression may serve as a predictive biomarker to target ME1. In sum, this biomarker-driven approach of deploying ME1 inhibitors in ME2 null and/or low expression cancers would be applicable in >25,000 patients in the US annually.

A chief concern with metabolism-targeted therapies is off-target toxicity. It was found that normal human pancreatic cells and fibroblasts tolerate ME1 inhibition. Further, germline Me1 knockout mice were developed. These are born and mature to old age (>2 years) without observable deleterious phenotypes. Similarly genetically engineered mice were created where we can knockout Me1 from the whole body of an adult animal, an experimental model that more closely mimics ‘drug’ treatment. These animals similarly do not exhibit adverse phenotypes, while having a fortuitous enrichment in CD8+ T cells in the lymph nodes. Finally, tumors transplanted into Me1 knockout animals confirmed the increase in T cell infiltration in tumors. These results suggest that ME1 inhibition may simultaneously target tumor cells and enhance anti-tumor immunity.

### Pancreatic Ductal Adenocarcinoma (PDA)

Pancreatic Ductal Adenocarcinoma (PDA) is an extremely aggressive disease. The 5-year survival rate is 10%, a number that has hardly increased in the last 30 years (Siegel et al. 2018). This owes in large part to the fact that effective treatment options for PDA do not exist. The physiology and biochemical nature of pancreatic tumors is fundamental to this therapeutic resistance. Specifically, pancreatic tumors are dense, avascular and highly inflammatory (Halbrook et al. 2017; Whatcott et al. 2015). Cancer cells in this environment are under severe physical and oxidative stress, and do not have functional vasculature to deliver oxygen and nutrients (Feig et al. 2012). Despite these unfavorable circumstances for growth, the cancer cells in these tumors thrive. Predictably, metabolic processes are altered in PDA cells facilitate survival and proliferation (Halbrook et al. 2017).

It was previously shown that mutant KRAS signaling is a major regulator of the reprogrammed metabolic state in PDA (Halbrook et al. 2017; Son et al. 2013; Ying et al. 2012; DeNicola et al. 2011). Among the pathways mediated by KRAS are those that protect cells from reactive oxygen species (ROS), a byproduct of metabolism. Excess ROS have the potential to be toxic to cells if their levels are not tightly regulated. PDA cells activate transcriptional and metabolic antioxidant pathways to tolerate the high rate of ROS generation, leading to an elevated ROS flux (Son et al. 2013; DeNicola et al. 2011). Together, this leads to both the over-utilization and dependence on antioxidant pathways to maintain redox balance, presenting metabolic vulnerabilities that may be therapeutically exploitable.

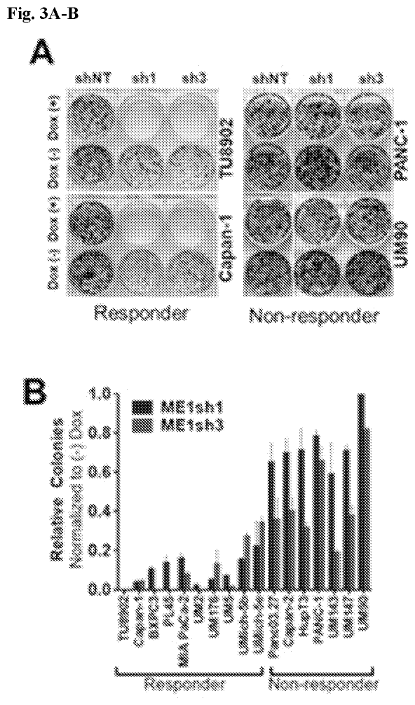

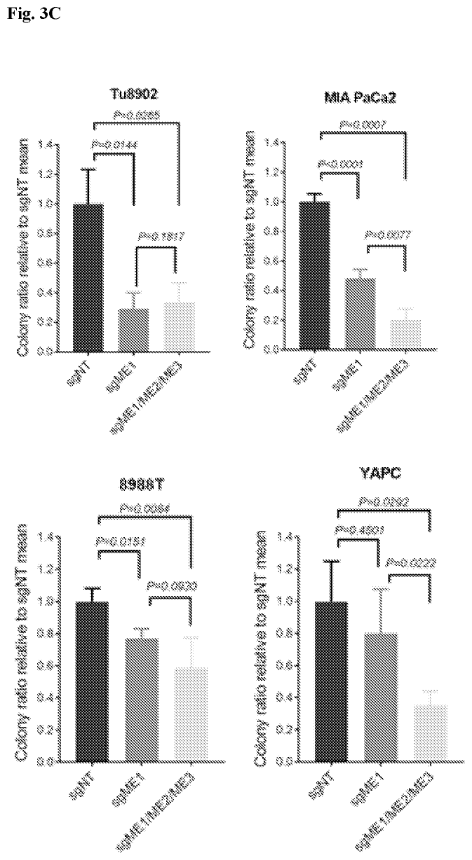

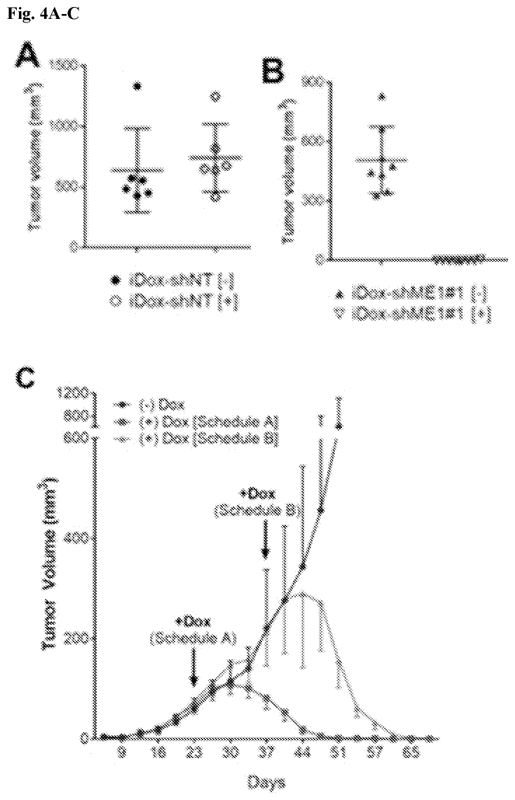

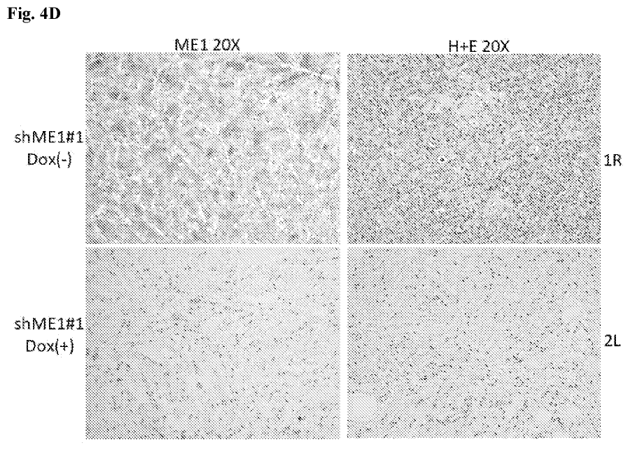

A ROS detoxification pathway in PDA that generates NADPH to maintain GSH pools has been described (Son et al. 2013). In this pathway, aspartate is synthesized in the mitochondria and released into the cytosol where it is acted on by a series of enzymes to ultimately yield NADPH from ME1. Genetic inhibition of ME1 leads to an increase in ROS, a drop in GSH, and pronounced suppression of PDA growth in vitro and in vivo (FIGS. 3-4). In contrast to ME1 knockdown in PDA cells, ME1 inhibition is well tolerated by normal human fibroblasts and pancreatic epithelial cells. Moreover, germline ME1 knockout mice were developed and it was found that these animals are born at Mendelian ratios, mature to adulthood, and are without observable phenotype. Additionally, ME1 null mice breed and produce litters, which as of this report have matured to adulthood and are similarly without discernable phenotype. In vivo tumor data in human patient-derived lines together with the viability of the knockout mouse model indicate that a desirable therapeutic window exists to target ME1.

### EXAMPLES

The disclosure now being generally described, it will be more readily understood by reference to the following examples which are included merely for purposes of illustration of certain aspects and embodiments of the present disclosure, and are not intended to limit the disclosure.

### Example 1. ME2 Compensates for ME1 Inhibition

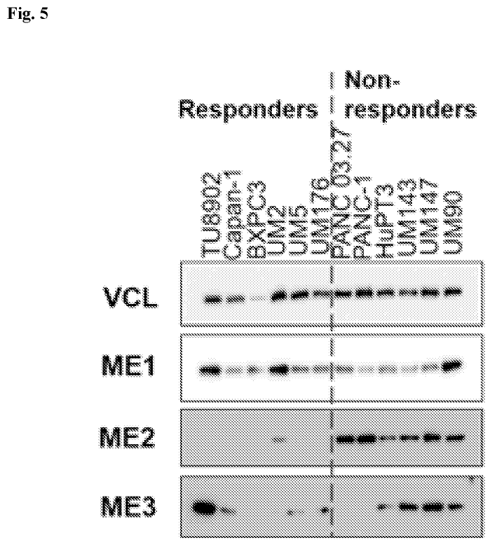

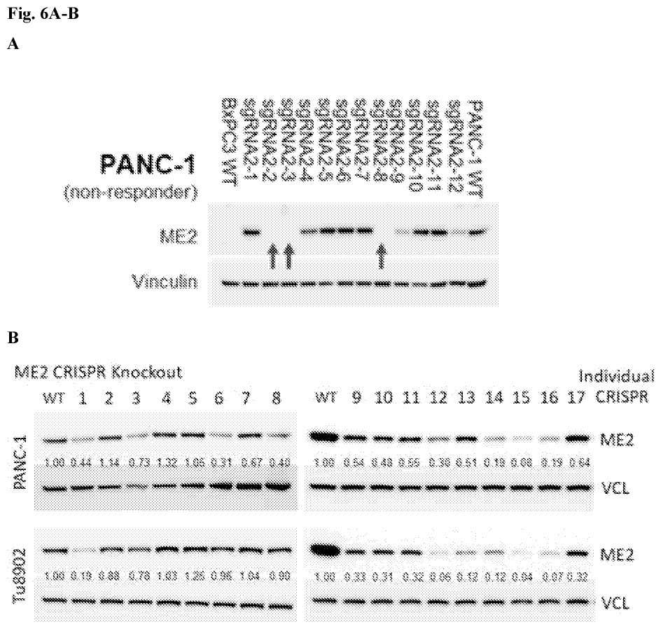

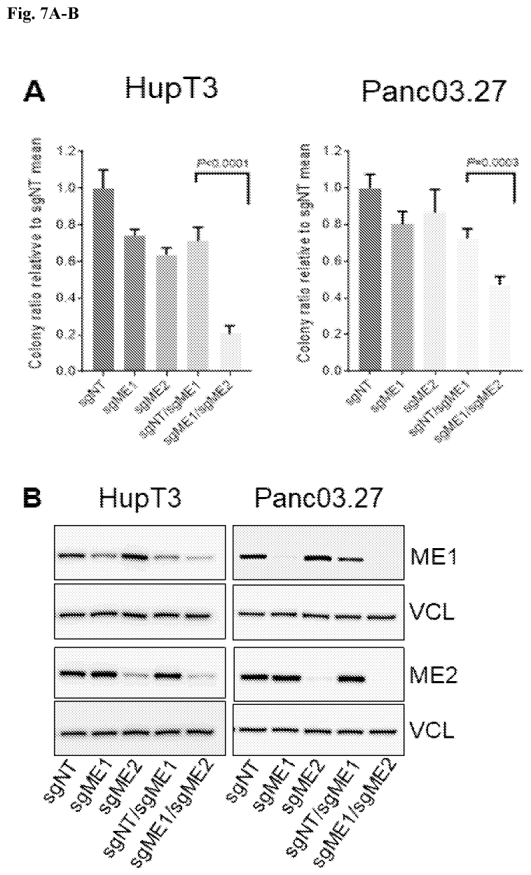

Based on initial observation of the inverse correlation between ME2 expression and ME1 knockdown sensitivity (FIG. 5), it was hypothesized that there is functional redundancy between ME1 and ME2. In order to test this hypothesis, clonal and pooled CRISPR/Cas9 ME2 knockout PANC-1 and TU8902 lines (FIG. 6) were generated under puromycin selection. PANC-1 cells represent our workhorse ME1 inhibition non-responsive line. This CRISPR/Cas9 approach is further extended to 3 PDA lines intermediate and 3 resistant to ME1 inhibition. ME2 knockout cell lines have been engineered under blasticidin or hygromycin selection and transduced with iDox-shNT (control) or iDox-shME1 or sgME1 to test the hypothesis that removing ME2 sensitizes PDA cells to ME1 inhibition. These cultures are subjected to ME1 inhibition and colony formation is analyzed, as in FIG. 3. FIG. 7 demonstrated supportive results.

### Example 2. Mechanistic Role of ME1

To determine the functional role(s) of ME1 in PDA, RNA-Seq analysis was performed with 7 PDA cell lines, including ME1 responder (i.e. TU8902, UM2, BxPC3, Capan-1), intermediate (i.e. TU8988T) and non-responder cell lines (i.e. PANC-1 and UM90) following ME1 knockdown (+Dox). These were compared to their isogenic control (−Dox). Unsupervised hierarchical clustering of significant differentially regulated genes revealed that a considerable proportion of mitochondrial transcripts were down regulated upon ME1 knockdown. Additionally, unbiased Gene Set Enrichment Analysis (GSEA) revealed a signature for endoplasmic reticulum (ER) stress as being uniquely upregulated in the responder but not in the non-responder lines (data not shown). Taken together, we next decided to characterize mitochondrial physiology and ER stress upon ME1 knockdown.

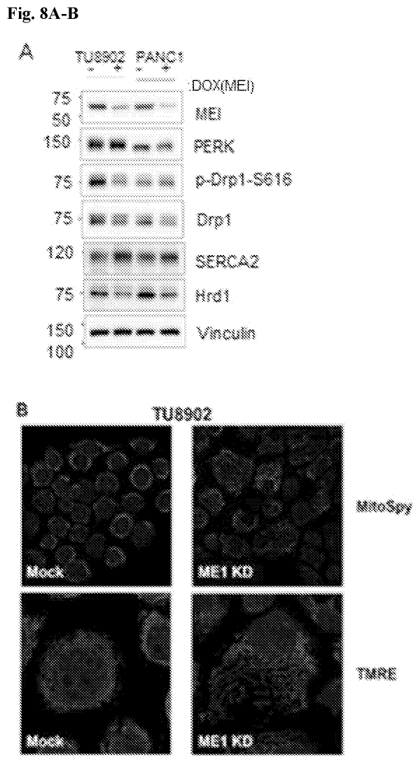

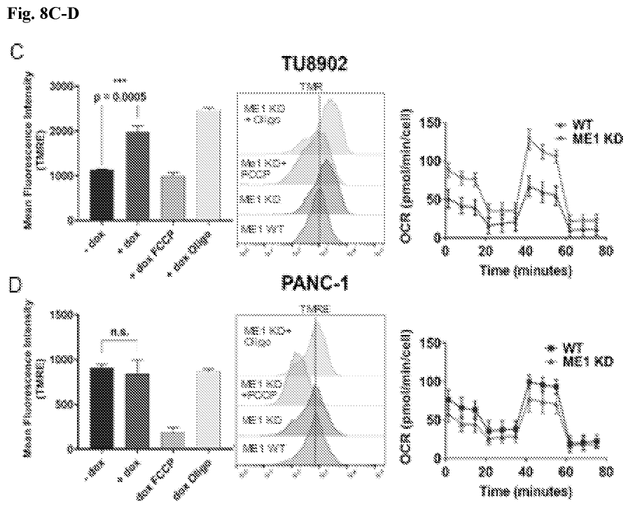

As indicated by Western blot, phospho-DRP1-S616, a biomarker for mitochondrial fission, is significantly down in ME1 responsive TU8902 following ME1 inhibition (FIG. 8A). Additionally, the increased level of SERCA2 and decreased level of Hrd1 upon inhibition of ME1 suggest that ER homeostasis is disrupted and that the unfolded protein response (UPR) pathway is activated. These results are consistent with the observed ER stress signature from the RNA-Seq data. To follow up on the down-regulated mitochondrial fission observed, we further performed confocal microscopy using live-staining mitochondrial dyes. Similar phenotypes were observed in two independent experiments using mitochondrial dyes either independent (MitoSpy, FIG. 8B, top) or dependent (TMRE, FIG. 8B, bottom) on mitochondrial membrane potential. Specifically, the mitochondria appeared to be more elongated and spread-out across the cell following ME1 inhibition. It was further observed that the TMRE signal to be increased significantly in TU8902 upon ME1 knockdown, indicating the presence of more polarized mitochondria (FIG. 8C, left and middle panel). Additionally, Seahorse measurement of oxygen consumption rate (OCR) further confirmed the presence of polarized mitochondria with increased OCR upon ME1 knockdown (FIG. 8C, right). In contrast, decreased Drp-1 phosphorylation (FIG. 8A) was not observed and increased mitochondrial membrane polarization or OCR (FIG. 8D) in PANC-1 upon ME1 knockdown was not observed.

### Example 3. Impact of Adult Me1 Loss on Mouse Behavior and Physiology

A cohort of 12 Me1 adult conditional knockout (cKO) mice, engineered to express a tamoxifen-inducible ubiquitin-Cre driven excision of the floxed Me1 allele (Me1^{f/f}), and 12 control Me1^{fl/fl} of equal sexes were aged to adulthood (8-12 weeks) and treated with tamoxifen to induce whole body Me1 knockout. These mice were weighed, aged to a year and closely monitored for basic behavioral analyses in their home cage. Changes in gait, mobility, or grooming patterns were not observed nor any unusual or bizarre behavior including, but not limited to, head licking, head searching, compulsive biting or licking, self-mutilation, circling and walking backwards. End point analysis of peripheral blood and gross morphological changes to the major organ systems again revealed no consistent or significant alterations. In total, this analysis is revealed no notable changes upon adult knockout of Mel that should be considered during the future development of ME1 inhibitors.

### Example 4. Allosteric Site ME1 Inhibitors

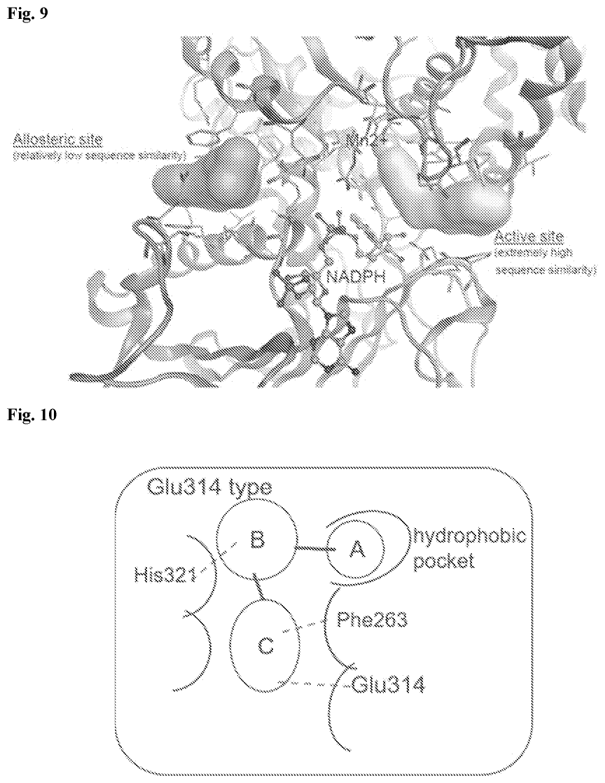

Compound 1-4 were tested (Table 1) for inhibition of ME1. It was determined that each of these compounds are allosteric site ME1 inhibitors (FIGS. 9 and 10). Capan1 is an ME1 responder line. H23 is an ME1 non-responder line.

TABLE 1

| | ME1 | MIE3 | CAPAN1 | H23 | Permeability |
|---|---|---|---|---|---|
| | IC₅₀/EC₅₀ | IC₅₀/EC₅₀ | IC₅₀/EC₅₀ | IC₅₀/EC₅₀ | pH 6.5 (×10⁻⁶ |
| | (μM) | (μM) | (μM) | (μM) | cm/sec) |
| 1 | 0.08 | 2.1 | 16.1 | 8.1 | 26.5 |
| 2 | 0.01 | 17 | 3.1 | 8.6 | <0.3 |
| 3 | 0.11 | NT | 26.6 | 4.8 | >30 |
| 4 | 0.58 | 16 | 1.8 | NT | >30 |

### Example 5. Active Site ME1 Inhibitors (Malate Competitive)

Compound 5-7 were tested (Table 2) for inhibition of ME1. It was determined that each of these compounds are active site ME1 inhibitors. Capan1 is an ME1 responder line. H23 is an ME1 non-responder line.

TABLE 2

| | ME1 | ME3 | CAPAN1 | H23 | Permeability |
|---|---|---|---|---|---|
| | IC₅₀/EC₅₀ | IC₅₀/EC₅₀ | IC₅₀/EC₅₀ | IC₅₀/EC₅₀ | pH 6.5(×10⁻⁶ |
| | (μM) | (μM) | (μM) | (μM) | cm/sec) |
| 5 | 4.1 | 53.00 | 2.4 | 8.6 | 15.1 |
| 6 | 1.8 | 77.0 | 0.78 | 1.7 | 18.1 |
| 7 | 7.4 | NT | 26.6 | 5.7 | NT |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification and the claims below. The full scope of the disclosure should be determined by reference to the claims, along with their full scope of equivalents, and the specification, along with such variations.

### REFERENCES

Brahmer, J.R. et al. Safety and activity of anti-PD-L¹ antibody in patients with advanced cancer. N Engl J Med. 2012;366(26):2455-65. Epub 2012/06/05.
Commisso, C. et al. Macropinocytosis of protein is an amino acid supply route in Ras-transformed cells. Nature. 2013;497(7451):633-7.
Cox, A.D.et al. Drugging the undruggable RAS: Mission possible? Nat Rev Drug Discov. 2014;13(11):828-51. Epub 2014/10/18.
DeNicola, G.M. et al. Oncogene-induced Nrf2 transcription promotes ROS detoxification and tumorigenesis. Nature. 2011;475(7354):106-9.
Feig, C. et al. The pancreas cancer microenvironment. Clin Cancer Res. 2012;18(16):4266-76. Epub 2012/08/17.
Halbrook, C.J. et al. Employing Metabolism to Improve the Diagnosis and Treatment of Pancreatic Cancer. Cancer Cell. 2017;31(1):5-19.
Halbrook, C.J. et al. Tumor cross-talk networks promote growth and support immune evasion in pancreatic cancer. Am J Physiol Gastrointest Liver Physiol. 2018;315(1):G27-G35. Epub 2018/03/16.
Muller, F.L. et al. Collateral Lethality: A new therapeutic strategy in oncology. Trends Cancer. 2015: 1(3); 161-173.
Rahib, L. et al. Matrisian LM. Projecting cancer incidence and deaths to 2030: the unexpected burden of thyroid, liver, and pancreas cancers in the United States. Cancer Res. 2014;74(11):2913-21. Epub 2014/05/21.
Siegel, R.L. et al. Cancer statistics, 2018. CA Cancer J Clin. 2018;68(1):7-30.
Son, J. et al. Glutamine supports pancreatic cancer growth through a KRAS-regulated metabolic pathway. Nature. 2013;496(7443):101-5.
Viale, A. et al. Oncogene ablation-resistant pancreatic cancer cells depend on mitochondrial function. Nature. 2014;514(7524):628-32.
Whatcott, C.J. et al. Desmoplasia in Primary Tumors and Metastatic Lesions of Pancreatic Cancer. Clin Cancer Res. 2015;21(15):3561-8.
Ying, H. et al. Oncogenic Kras maintains pancreatic tumors through regulation of anabolic glucose metabolism. Cell. 2012;149(3):656-70.
Yoshida, T. et al. Discovery and Characterization of a Novel Allosteric Small-Molecule Inhibitor of NADP+-Dependent Malic Enzyme 1. Biochemistry. 2022: 61(15); 1548-1553.

We claim:

1. A compound of formula (IA): 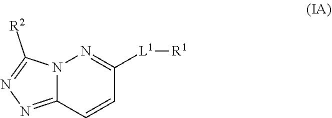
wherein
L¹ is a single bond connecting to R¹, or is —C(O)NH— or —NHC(O)—;
R¹ is aryl, heteroaryl, or heterocyclyl; and
R² is aryl, heteroaryl, or heterocyclyl;
or a pharmaceutically acceptable salt thereof.

2. The compound of claim 1, wherein L¹ is a single bond to R¹.

3. The compound of claim 2, wherein R¹ is substituted heterocyclyl.

4. The compound of claim 3, wherein R¹ is an alkyl substituted piperidinyl.

5. The compound of claim 4, wherein R¹ is

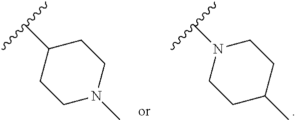

6. The compound of any one of claims 1-5, wherein R² is substituted aryl or heteroaryl.

7. The compound of claim 6, wherein R² is a phenyl substituted with at least one R^{2a} selected from halo, alkylamino, alkylaminoalkyl, and —NHC(O)R^{2b}, wherein R^{2b} is a substituted heterocyclyl.

8. The compound of claim 6 or 7, wherein R² is a phenyl substituted with one R^{2a} selected from alkylaminoalkyl and —NHC(O)R^{2b}, wherein R^{2b} is a substituted heterocyclyl.

9. The compound of claim 7 or 8, wherein R^{2b} is an alkyl substituted piperidinyl.

10. The compound of any one of claims 1-9 having the structure: 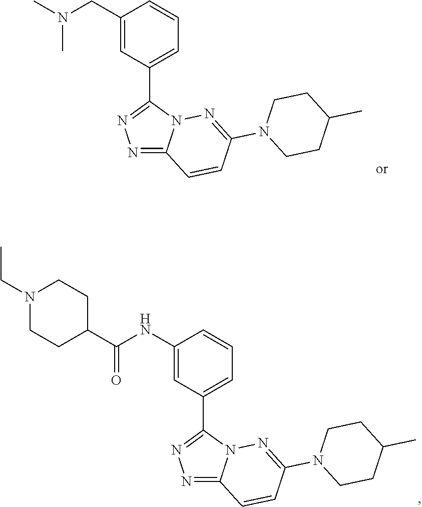
or a pharmaceutically acceptable salt thereof.

11. A compound of formula (IB): 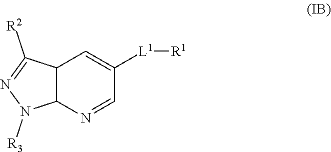
wherein
L¹ is a single bond connecting to R¹, or is —C(O)NH— or —NHC(O)—;
R¹ is aryl, heteroaryl, or heterocyclyl;
R² is alkyl, aryl, heteroaryl, or heterocyclyl; and
R³ is aryl or heteroaryl,
or a pharmaceutically acceptable salt thereof.

12. The compound of claim 11, wherein L¹ is —C(O)NH—.

13. The compound of claim 12, wherein R¹ is substituted heterocyclyl.

14. The compound of claim 13, wherein R¹is an alkyl substituted piperidinyl.

15. The compound of claim 14, wherein R¹ is

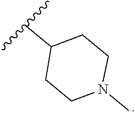 [SMILES: *C1CCN(C)CC1]

16. The compound of any one of claims 11-15, wherein R² is alkyl.

17. The compound of any one of claims 16, wherein R² is C₁-C₆ alkyl.

18. The compound of any one of claims 11-17, wherein R³ is substituted aryl or heteroaryl.

19. The compound of claim 18, wherein R³ is a phenyl substituted with at least one R^{3a} selected from halo, alkylamino, alkylaminoalkyl, and —NHC(O)R^{3b}, wherein R^{3b} is a substituted heterocyclyl.

20. The compound of claim 18 or 19, wherein R³ is a phenyl substituted with one R^{3a} selected from a halo.

21. The compound of claim 19 or 20, wherein the substituted phenyl is substituted at the 3 or 4 position of the phenyl group.

22. The compound of any one of claims 11-21 having the structure: 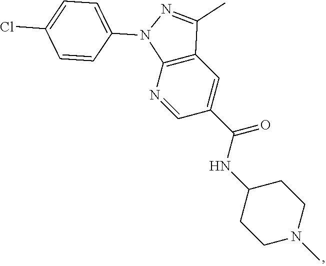 [SMILES: Cc1nn(-c2ccc(Cl)cc2)c2ncc(C(=O)NC3CCN(C)CC3)cc12]
or a pharmaceutically acceptable salt thereof.

23. A compound of formula (IC): 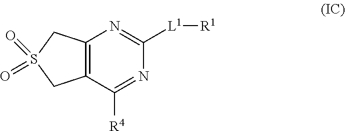
wherein
L¹ is a single bond connecting to R¹, or is —C(O)NH— or —NHC(O)—;
R¹ is aryl, heteroaryl, or heterocyclyl; and
R⁴ is a heterocyclyl,
or a pharmaceutically acceptable salt thereof.

24. The compound of claim 23, wherein L¹ is absent.

25. The compound of claim 24, wherein R¹ is substituted heterocyclyl.

26. The compound of claim 25, wherein R¹ is an alkyl substituted piperidinyl.

27. The compound of claim 26, wherein R¹ is

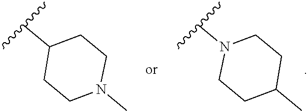

28. The compound of any one of claim 23-27, wherein R⁴ is a fused bicyclic heteroaryl.

29. The compound of claim 28, wherein R⁴ is a [6.5] fused bicyclic heteroaryl comprising at least one nitrogen.

30. The compound of claim 28 or 29, wherein R⁴ is a [6.5] fused bicyclic heteroaryl comprising three nitrogens.

31. The compound of claim 30, wherein R⁴ is

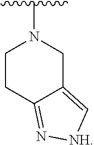 [SMILES: *N1CCc2n[nH]cc2C1]

32. The compound of any one of claims 23-31 having the structure: 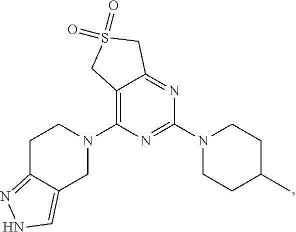 [SMILES: CC1CCN(c2nc3c(c(N4CCc5n[nH]cc5C4)n2)CS(=O)(=O)C3)CC1]
or a pharmaceutically acceptable salt thereof.

33. A compound of formula (II) or a pharmaceutically acceptable salt thereof: 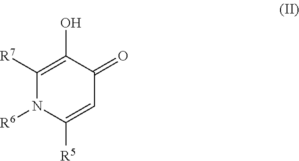
wherein
R⁵ is alkyl;
R⁶ and R⁷ are, independently, H, alkyl, -L²-L³-R⁸, or -L²-R⁸;
R⁸ is aryl, heteroaryl, or heterocyclyl;
L² is alkylene or heterocyclyl; and
L³ is heterocyclyl;
provided that one and only one of R⁶ and R⁷ is -L²-L³-R⁸ or -L²-R⁸.

34. The compound of claim 33, wherein the compound is not

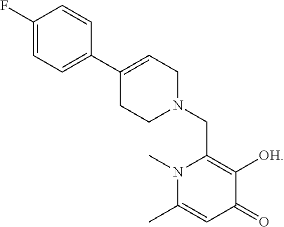 [SMILES: Cc1cc(=O)c(O)c(CN2CC=C(c3ccc(F)cc3)CC2)n1C]

35. The compound of claim 33 or 34 having the structure: 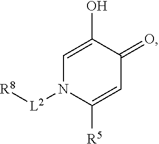
or a pharmaceutically acceptable salt thereof.

36. The compound of claim 35, wherein R⁵ is C₁-C₆ alkyl.

37. The compound of claim 36, wherein R⁵ is —CH₃.

38. The compound of any one of claims 35-37, wherein L² is C₂-C₆ alkylene.

39. The compound of any one of claims 35-38, wherein R⁸ is a fused bicyclic heteroaryl.

40. The compound of claim 39, wherein R⁸ is an [6.6] fused bicyclic heteroaryl comprising at least one nitrogen.

41. The compound of claim 40, wherein R⁸ is

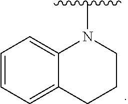 [SMILES: *N1CCCc2ccccc21]

42. The compound of any one of claims 35-38, wherein R⁸ is substituted aryl or heteroaryl.

43. The compound of claim 42, wherein R⁸ is a phenyl substituted with at least one R^{8a} selected from halo, alkylamino, alkylaminoalkyl, and —NHC(O)R^{8b}, wherein R^{8b} is a substituted heterocyclyl.

44. The compound of claim 43, wherein R⁸ is a phenyl substituted with one R^{8a} selected from a halo.

45. The compound of claim 43 or 44, wherein the substituted phenyl is substituted at the 3 or 4 position of the phenyl group.

46. The compound of claim 33 having the structure: 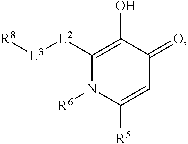
or a pharmaceutically acceptable salt thereof.

47. The compound of claim 46, wherein R⁵ is C₁-C₆ alkyl.

48. The compound of claim 47, wherein R⁵ is —CH₃.

49. The compound of any one of claims 46-48, wherein R⁶ is C₁-C₆ alkyl.

50. The compound of claim 49, wherein R⁶ is —CH₃.

51. The compound of any one of claims 46-50, wherein L² is C₁-C₆ alkylene.

52. The compound of any one of claims 46-51, wherein L³ is -piperidinyl- or -tetrahydropyridinyl-.

53. The compound of claim 52, wherein L³ is

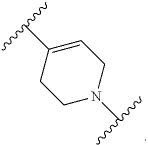 [SMILES: *C1=CCN(*)CC1]

54. The compound of any one of claims 46-53, wherein R⁸ is substituted aryl or heteroaryl.

55. The compound of claim 54, wherein R⁸ is a phenyl substituted with at least one R^{8a} selected from halo, alkylamino, alkylaminoalkyl, and —NHC(O)R^{8b}, wherein R^{8b} is a substituted heterocyclyl.

56. The compound of claim 55, wherein R⁸ is a phenyl substituted with one R^{8a} selected from a halo.

57. The compound of claim 55 or 56, wherein the substituted phenyl is substituted at the 3 or 4 position of the phenyl group.

58. The compound of any one of claims 33-45 having the structure 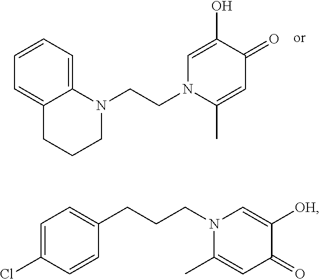 [SMILES: Cc1cc(=O)c(O)cn1CCN1CCCc2ccccc21 ; Cc1cc(=O)c(O)cn1CCCc1ccc(Cl)cc1]
or a pharmaceutically acceptable salt thereof.

59. The compound of claim 33 or 46-57 having the structure 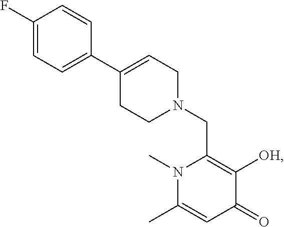 [SMILES: Cc1cc(=O)c(O)c(CN2CC=C(c3ccc(F)cc3)CC2)n1C]
or a pharmaceutically acceptable salt thereof.

60. A pharmaceutical composition comprising a compound of any one of claims 1-59 and one or more pharmaceutically acceptable excipients.

61. A method of treating a cancer in a subject in need thereof, the method comprising administering a therapeutically effective amount of the compound of any one of claims 1-59 to the subject.

62. The method of claim 61, wherein the cancer is mediated by malic enzyme 1 (ME1).

63. The method of claim 61 or 62, wherein the cancer is a malic enzyme 2 (ME2)-deficient cancer.

64. The method of any one of claims 61-63, wherein the cancer is a gastrointestinal cancer.

65. The method of claims 64, wherein the gastrointestinal cancer is esophageal cancer, stomach cancer, colorectal cancer, pancreatic cancer, or liver cancer.

66. The method of claims 64, wherein the gastrointestinal cancer is pancreatic ductal adenocarcinoma (PDA).

67. A method of treating a cancer in a subject in need thereof, the method comprising administering to the subject a therapeutically effective amount of an agent that downregulates the copy number, amount, and/or activity of ME1.

68. The method of claim 67, wherein the agent is a CRISPR guide RNA (gRNA), RNA interfering agent, antisense oligonucleotide, peptide or peptidomimetic inhibitor, aptamer, antibody, or intrabody.

69. The method of claim 68, wherein the RNA interfering agent is a small interfering RNA (siRNA), CRISPR RNA (crRNA), a small hairpin RNA (shRNA), a microRNA (miRNA), or a piwi-interacting RNA (piRNA).

70. The method of claim 68, wherein the RNA interfering agent is a CRISPR guide RNA (gRNA).

71. The method of claim 68, wherein the agent comprises an antibody and/or intrabody, or an antigen-binding fragment thereof, which specifically binds to ME1.

72. The method of any one of claims 67-71, wherein the cancer is mediated by ME1.

73. The method of any one of claims 67-72, wherein the cancer is a malic enzyme 2 (ME2)-deficient cancer.

74. The method of any one of claims 67-73, wherein the cancer is a gastrointestinal cancer.

75. The method of claims 74, wherein the gastrointestinal cancer is esophageal cancer, stomach cancer, colorectal cancer, pancreatic cancer, or liver cancer.

76. The method of claims 74, wherein the gastrointestinal cancer is pancreatic ductal adenocarcinoma (PDA).
